(19) United States
(12) Patent Application Publication
(10) Pub. No.: US 2018/0021224 A1
(43) Pub. Date: Jan. 25, 2018

(54) Pacifier Holder ("PH") & Eye Cover ("EC" attachment)

(71) Applicant: Mike Samadi, Evans, GA (US)

(72) Inventor: Mike Samadi, Evans, GA (US)

(73) Assignee: Morgan Mojgan Samadi, Evans, GA (US)

(21) Appl. No.: 15/214,520

(22) Filed: Jul. 20, 2016

(51) Int. Cl. A61J 17/00; A61F 9/04

(52) U.S. Cl. CPC A61J 17/001; A61F 9/04

(57) ABSTRACT

As a result of the repeated incidents (described in the Background section), the inventor decided to create a layer of soft cushioned strap that would allow the pacifier gently stay in a baby's mouth until the person holding (in charge of caring) or the baby decides to remove it. This creates a sanitary method of continuous satisfaction for the baby, which in turn reduces baby's crying—wanting a support, substitute nourishment, or a method to keep the baby busy, rested and unstressed. The invention may be in four (4) design types, using the same material but creating a variable styles (i.e. PH, PHB, PHC and PHD) as shown in Specification section.

### BACKGROUND FOR THE INVENTION

The inventor just become a proud father. Through the first few weeks of nursing the baby, he learned that she (the baby) becomes frantic and frustrated if the pacifier is moved or falls out of her mouth. It was also observed that sometimes as soon as pacifier becomes loose (but still in her mouth), she begins to cry. The crying occurs a lot until the pacifier is placed back in her mouth.

In general, a pacifier serves several purposes including calm the restless and crying baby, hold the hungry baby for a little longer until she is fed, allows the baby fall sleep quicker and many more. However, the baby would accidentally and unconscionably drop the pacifier (sometimes, it may even fall on the floor). By the time it is placed back in her mouth, the baby would cry frantically and gets severely stressed. This situation made the parent think of a method in which the pacifier could be kept in the baby's mouth as needed.

In two days-cares, inventor observed that the care-provider wrapped a baby blanket around the mouth and behind the baby's head/neck in an attempt to hold the pacifier in place. This action is not only dangerous, since a slit move by the baby, would cause the blank to move over his/her nose, blocking baby's breathing path (causing suffocation), and would hurt the back of baby's neck since the head and neck is not laid flat (as the nature intended). Therefore, the idea of Pacifier Holder/Pacifier Nanny, Pacifier Strap/Harness (hereinafter “PH”) was born.

### CURRENT STATE OF ART

After a full search for similar development of this design, the inventor of this Pacifier Holder was able to find only one design that may compete with this invention. However, that invention (U.S. Pat. No. 4,969,894—Pub date: Nov. 13, 1990) has flaws. That invention deforms the shape of baby's ears (pushing them forward and causing the ears to stick out).

This invention PH does not create that problem. Moreover, the invention of 1990 has not been manufactured nor marketed. But the intent for PH is to immediately manufacture and market upon granting of the patent.

### BRIEF SUMMARY OF INVENTION

The present invention relates to the concept of having a pacifier holder strap or harness, in particular a stretchable retainer to very gently and yieldably hold a pacifier gently in a baby/infant mouth. The stretchable retainer consists of a thin, flexible, stretchable, soft strip of fabric (varieties of designs and shapes) with a preferated hole in its center to hold a pacifier and:

a. velcro at each end to interlock and securely but yield ably close a gentle circle around the baby's head and face, or

b. a thin, soft and stretchable elastic, formed in circular figure, at each end of the strap/harness, acting as ear-receiving aperture to receive a child's ear so that each anchoring end can be quickly and easily attached to the child in an attempt to hold the pacifier in the baby's mouth.

The elastic strap is threaded through both of apertures to couple the pacifier to pacifier-supporting strap and to arrange the two anchoring members (either item a. or b.—above) in spaced-apart relation straddling the pacifier.

One major Advantage is that the pacifier retainer/strap is made of materials that is so flexible and stretchable that if desired or necessary, could be removed by the infant/baby without assistance and would allow the baby to easily breath, cry, smile or even make noise without gagging.

There are other advantages, such as, it fits any size pacifier, is very light weight, portable on baby's face anywhere, and adjustable to fit any size baby.

Benefit Statements:

A safe way of continuous use of pacifier by

0001. reducing a newborn baby's cries and stress level,

0002. reducing baby's refluxed,

0003. increasing baby's digestive activity,

0004. helps baby itching and scratching gum issues during the early stage of teething

0005. assuring the parents or the care giver that the baby is not stressed or restless,

0006. assisting on baby's continuous gum development even at the time the baby is sleep,

0007. giving parents of new baby an extended rest so that they do not have to run back and forth to place pacifier in a crying baby's mouth; and

0008. protecting a baby from inserting other objects that may also be unsanitary into her mouth.

0009. with this invention, if a pacifier falls out of baby's mouth it is kept close to his/her mouth and would not fall on ground/floor, where it would make child could also be exposed to an unsanitary pacifier unless the pacifier is cleaned before it is put back into baby's mouth.

0010. One major Advantage is that the pacifier retainer/strap is made of materials that is so flexible and stretchable that if desired or necessary, could be removed by the infant/baby without assistance and would allow the baby to easily breath, cry, smile or even make noise without gagging.

0011. It fits any size pacifier

0012. It is very light weight, portable on baby's face anywhere, and

0013. It is adjustable to fit any size baby.

### BRIEF DESCRIPTION OF DRAWINGS

FIG. 1 is a completed and frontal view of the final product of one of the several ways the Pacifier Holder (PH)/Nanny shall look. The fold on each side of the center means that the length of the Pacifier Holder (PH), Pacifier Nanny Stretches is is long.

FIG. 2 is a completed and frontal view of FIG. 1; however, it provides detail of some of the parts of the invention as well as some size requirements for the PH strap. Moreover, for simplicity, the folded area/length of FIG. 1 is not displayed (in this and ALL subsequent figures below), but is assumed the length (in this and all figures below) to be the same exact length as FIG. 1.

FIG. 3 is a completed back view of FIG. 1, again—assuming this is the same length as FIG. 1 (even though the folds are not displayed), showing other connecting parts (velcros) of retainer strap and their sizes in relation to the entire PH. The Velcros cause the two end of the PH strap to connect in the back of the baby's head.

FIG. 4 is a side view of the FIG. 1.

FIG. 5 is a completed view of the FIG. 1, again—assuming this is the same length as FIG. 1 (even though the fold is not displayed), showing additional design—where the baby's eye-cover would be attached—through the Velcro strips on the upper part of the PH strap

FIG. 6 is size and full diagram of the eye cover in which would be attached to the upper center part of the FIG. 5 to complete the eye-cover of the PH.

FIG. 7 is a different design of the PH strap where the length of the PH strap is longer on one side, causing the retainer Velcro be attached on the side/cheek of the baby's face—where FIG. 3 shows that both sides of the PH strap is the same length causing the two ends of the strap to connect behind the baby's head.

FIG. 8 is another style of the PH strap. In this design, the use of velcro was replaced by the elastic loop on each end to fit the baby's ears. In this design, the length of the PH strap is much shorter, since the PH strap docs not loop around the baby's head and instead, each end is held by going around the baby's ears.

### DESCRIPTION OF INVENTION

As a result of the repeated incidents (described in the Background section), the inventor decided to create a layer of soft cushioned strap that would allow the pacifier gently stay in a baby's mouth until the person holding (in charge of caring for) the baby decides to remove it. This creates a sanitary method of continuous satisfaction for the baby, which in turn reduces baby's crying—wanting a support, substitute nourishment, or a method to keep the baby busy.

Babies get stressed easy. Babies have three ways to get stressed and cry, it could be they are hungry, need changing or sleepy. The pacifier can provide temporarily replacement for hunger and is a good support to put baby to sleep quickly. However, babies lose control of pacifier and it falls out of their mouths. When it falls out, they get extremely stressed and start crying. Thus, the best and only way to keep their mouths shut is that a pacifier is kept in their mouths.

This is a novel method of reducing partners or care-giver repeated and frequent rush to baby's side in order to place pacifier in her mouth and reduce her uneasiness. This method will also reduce accidents since the care-giver may carelessly cause an accident while rushing to baby's help.

This invention may be design in four (4) different ways/styles using the same concept (a Pacifier Holder “PH” Pacifier Nanny), using the same material but creating a variable styles (i.e. PH, PHB and PHC—as shown in the diagram section or detailed in the Specification section).

This invention relates to a Pacifier Holder, pacifier strap/harness (a.k.a “PH strap”). It is a stretchable retainer for yieldably holding a pacifier gently in a baby's mouth. This will also reduce the risk of a child be exposed to an unsanitary pacifier (fallen on ground or other surfaces, to include unclean cloth) unless the pacifier is cleaned promptly before it is back in the child's mouth.

The objective here is to present a method or item by providing a strap, harness or other means for a very gentle and yielding pacifier in a child's mouth (who still desires a pacifier). Yet, this method should be applied in a manner that the baby would not feel any tension, force, weight or stress as a result of having such a strap over his/her head, face and neck.

Moreover, this invention would be made of fully non-metallic or other harmful material (even when the cover is exposed or torn) so that at no time would it create a danger to the child desiring a pacifier. The non-metallic elements will also assist when the baby passes a security area, human scanner or other security devices (will not activate the scanning device).

### How to Make Pacifier Holder (PH)/Nanny

### Detailed Description of Invention

There are varieties of ways to make this invention. The final purpose of this invention is to have a strap gently warped around a baby's mouth, back of head and/or neck, so that it would hold the pacifier in place (in mouth) until it is desired to be removed by the nursing/controlling person.

Items Used in this Invention are:
a. A soft fabric (preferably thin, cotton),
b. Elongated elastic strap or stretchable gentle rubber,
c. Velcro or washable sticky tapes (that won't lose its stickiness after being washed),
d. Needle and thread (a sewing machine),
e. A soft thin rope style link to make the end loop (for clipping/attaching to baby's cloth),
f. A pacifier, a pencil/pen, and thin round elastic for the design of second method of PHD,
g. A good caring tailor.

Process of Constructing this Invention:

0001. Cut a strip of 14-17″×2-4″ inches of soft fabric (describe in item a. above).

Note 1: The length of the fabric being used should be 1.5-2.5 inches longer than the length of the elastic strap/rubber being used (as described in item 2—below).
Note 2: The width of the fabric being used should be 2.5 times the width of strap/rubber. This is due to any adjustment and final surging of the lower edge of fabric. In addition, it allows the fabric being folded correctly over to the width of the strap (perfectly covering/wrapping the strap/rubber inside the fabric).
Note 3. These measurements depend on the size of baby's head & neck and age. Therefore, the final design will have 2-3 variation of length (small, medium and large), so that it would fit tinny baby's as well as babies with full cheeks, soft double chin and thick neck.
Note 4: Reemphasizing Note 1. The strap/rubber should be 1.5-2.5 inches shorter in length than the fabric. Therefore, as the strap/rubber being laid over fabric, there should be 0.75″-1.25″ excess fabric on each end (left & right) of the strap/rubber.

0002. Understanding the notes (1-4) above, cut a 12.5-14″ elastic strap/rubber (described in item b. of “Item used . . . ”—sub-section above).

0003. Fold the fabric into half-width-wise (making it a narrower strip of fabric).

0004. Lay the elastic strap/rubber inside the fold of fabric, so that the top length of both items will meet (at the fabric's fold).

0005. Now, while holding both fabric and strap/rubber, bring the right edge of strap to close to right edge of fabric—but 0.25″ away from fabric's edge. Sew/stitch the right edge of strap to fabric.

0006. Now, holding the right side (which is sewed), stretch the strap/rubber to the left—inside the fabric until meets the other end of the fabric (but 0.25″ away from fabric's left edge). While strap being stretched almost to length of the fabric (−0.25″), sew/stitch the two to each other lengthwise (at the long-center of strap/rubber) sewing the two items together and run stitch again but about 0.25″ or more lower so that it would fully secure the strap/rubber to the fabric. Now, you have an almost complete stretchable fabric strap. The process is almost complete, except that the two lower edges of the fabric are still loose.

0007. Make certain that the two lower/bottom sides of the fabric are aligned and then run the strap through a surger (while stretching the fabric utilizing the full length of the fabric and the stretched rubber inside of it). The first phase of your “PH strap” is now complete.

Note 5: At this moment, you have NOT sewed the left side yet, because we have to insert the tip of the “ [symbol] ” hook/loop inside this newly combined strap (“PH” strap) before this side is sewed.

At this moment, you will have—as illustrated by FIG. 2 excluding the “ [symbol] ” on the top side, “◯” at the center, “ [symbol] ” on the left side and/or the velcro marking of FIG. 2.

0008. Now cut a 2-3″ strip of velcro (both male and female). (described in item c. of “Item used . . . ”—sub-section above)

0009. Flip the PH strap (described in parg. 6 & 7—above) (a.k.a. Pacifier Holder “PH” strap) so that the bottom view of the PH strap would be showing.

0010. Lay the female side of velcro on the left edge of the newly PH strap. Move it by 0.25″ away from the edge of strap towards the center (lengthwise).

0011. Also align this velcro vertically so that it would be placed vertically at the center (when considering the top and bottom edge—width-wise).

0012. Sew the female Velcro to the PH strap.

0013. Using the soft thin rope style link (described in item e. of “Item used . . . ”—sub-section above), cut a 0.75″ strip and insert the two ends inside the left side of PH strap (still unsewed) (making a loop with the rope at the end). Now sew the left side (keeping the “ [symbol] ” attached in place and tight). Please sew this side again a couple of times so that it would also hold “ [symbol] ”.

0014. Now flip the PH strap to the front side and repeat steps 10-12 (above) for the male portion of the velcro to be sewed to the right-side (opposite end of where female velcro was just sewed) of PH strap (but on the front side of the PH strap.

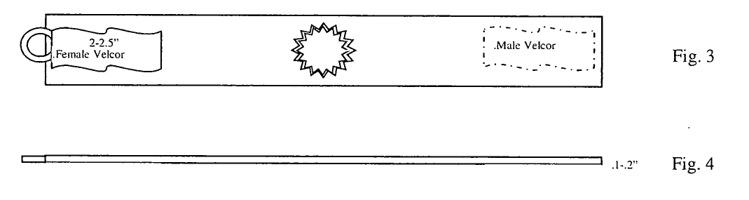

0015 Now, you are looking at the FIG. 3 (excluding the “◯” at the center).

0016. With practically all sides sewed fully, sew the PH strap lengthwise couple more times (while you stretch it fully so that the strap/rubber inside would be fully attached (joined) to the fabric surrounding it—to make a fully stretched, elastic consolidated strap. This could be done with the sewing machine on double or triple stitch mode and or elastic mode (to insure the strap remains flexible and elastic)

0017. To make a complete FIG. 3, measure the full stretched length of the strap. Then, measure the half-length and mark it. Now, mark the vertical (width) center of this strap. The exact center of the strap is now located and marked.

0018. Use a coin (the size of a USA—25 cent, a quarter) and place the center of the coin at the center of the strap. Then use the pencil/pen and draw a circle around the coin on the PH strap.

0019. Remove the coin and cut a circle as shown by “◯” (making a hole).

0020. Instead of steps 17-18 (above), a horizontal line of 1.0″ can be cut (slot) at the center of this PH strap to substitute for the circular hole (“◯”). This line would create a button hole on cloth. In this invention, instead of a button, the handle of pacifier would pass through this slot.

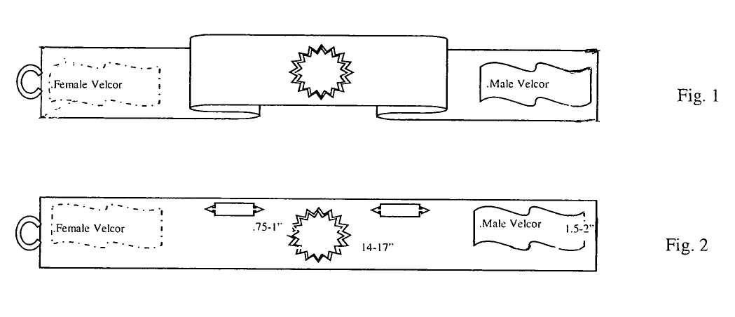

0021. Then use the sewing machine and stitch around this circle/slot, so that it will remain elastic and open for the end of the pacifier (handle) pass through. Now you will have FIG. 1.

0022. Insert the pacifier handle through this hole/slot. The hole/slot should hold the pacifier tight at the center of the PH strap.

Second, Third and Fourth Design Styles of this Invention: PHB, PHC & PHD:

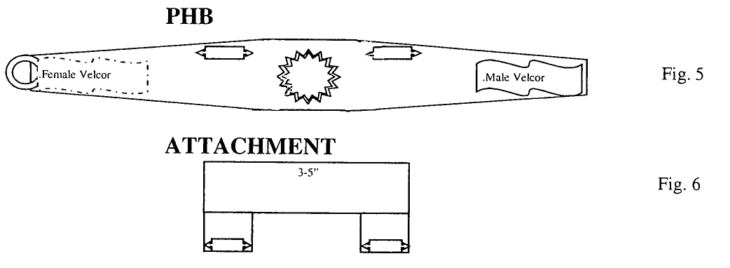

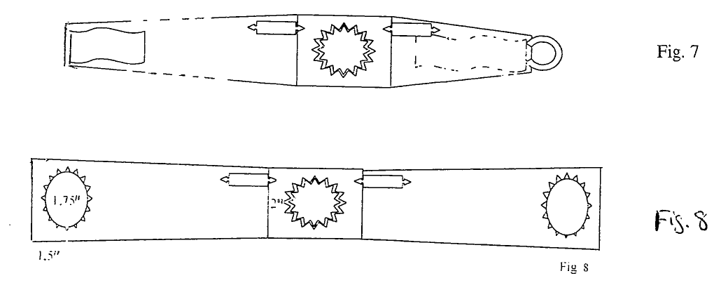

0023. A second, third and fourth inventions that would share the same description, problem resolution, design and methodology as PH—are illustrated in the diagram section by FIG. 5 and FIG. 7 (also referred to as PHB, PHC and PHD). These three provide variable style of the PH invention.

0024. PHB offers the same design as PH, but the end of the strap (“strap” is not as wide as the center. In this design, the ends of the straps are narrower by half width size of PH to reduce any tightness between the fabric and the baby's neck.

0025. In the PHC unit, one side of the strap (PH strap) is shorter than the other side by 6-8″ inches. i.e. If the entire length of the PH strap is 14″, then, one side of PH strap will have a length of 10-11″ and the other side will have the length of 4-3″. In this style, the male and female velcro (at the two ends of the PH strap), will meet and attach on the side of the baby's face (above the jaw line, below the cheek or ear). This is done so that the strap will not cause any discomfort (if any) to the back of the neck or lower side of the back of the head.

However, material used and process of making this will be similar to the actual PH strap (other than the length of left side of the strap would be different from right side by 6-8″. The attaching velcro can be on either side of the baby's face and the location of the hook/loop can also be adjusted to accommodate for it to be close to a button on baby's cloth.

0026. The PHD is also similar in design, however, instead of the strap to loop around the baby's neck/head, the ends loop around the baby's ears (as described in the diagram FIG. 8). In this design, the length of the fabric would be between 8-9″ (instead of the PH, PHB, PHC that they are 14-17″—depending on the size of baby's head, face and neck). In this design, the end of the fabric is cut inform of a circle of 1.5″ in diameter and the edge of the holes are double stitched. Now, instead of the PH strap to loop around the baby's head and neck, it will be looped around the baby's ears. Therefore, when PHD is complete, the baby's ears are gently closed/folded, then, the holes of PHD will go around the baby's ears. In this method, the end of the fabrics will rest gently and easily around the baby's ears, holding the Pacifier Holder/Nanny in place as it was supposed to.

0027. Meanwhile, PHD can also be designed in a manner that instead of having fabric to go around the baby's ears, a thin round elastic of 2-3″ can be sewed to the end of the strap and placed around the baby's ear. In this method, the length of the PH strap should be reduced to 5-7″. Then the thin elastic should be sewed to the two ends- to accommodate for the remaining length.

An Additional Part to this Design is Attachment (EYE Cover):

0028. Although the attachment is not necessary part of the PH, PHB, PHC, and PHD strap, but it can be added as part of them to protect the baby's eye (use as “Eye-cover”). This is important when baby is restless, wants to go to sleep, but either lights or something else (environment) bothers him/her and forces him/her to keep her eyes open (in desperation and distraction). It prevents the baby to use his/her hands over the eyes, hence reducing the risk of his/her fingernails scratch her/his eyes, eye-led or any part of his/her face). The inventor's baby and other similarly newborn babies in two day-cares, all seem to do the same, place their hands or fists over their eyes. Not coincidentally, every one of the babies had a scratch marks from their fingernails somewhere around their eyes-lids.

In three day-care centers, the inventor observed that the care providers covered the babies' faces with a baby-blank (in the absence of proper shade attachment to the babies' crib or rocking chair). Inventor was shocked and removed his baby from all day cares—knowing that an accident is bound to happen.

Construction of Attachment (Eye Cover):

0029. For constructing this part (Attachment), items a. and c. of the “Item used . . . ”—sub-section (above), would be needed.

0030. Cut a piece of the fabric 4-6″ in length and 3-4″ in width.

0031. Cut an opening as illustrated by FIG. 6 (attachment) to open space for baby's nose.

0032. Cut two sets of male & Female velcro or two pieces of washable sticky tape (this item is preferred).

0033. Sew one side of the sticky tape (or male velcro) to the top side of PH (above) on the left side and repeat the same on the right side (as illustrated by the symbols “ [symbol] ” in FIGS. 2 and 5).

0034. If the stick tape is used, skip this step. For velcro, sew the male side of the velcro to the attachment (as illustrated by the symbols “ [symbol] ” in FIG. 6).

Use of Attachment:

It is very simple/easy to use. Get an attachment, flip it so that the sticky tape (or the female velcro) side of the attachment meets male velcro at the top side of the PH strap (above the PH mouth hole area—as illustrated by “ [symbol] ” in FIGS. 2 and 5).

Note 6: If velcro is used, (prefer sticky/adhesive tape) then each of the “ [symbol] ” represents one side and piece of the velcro that is going to be sewed to the attachment or to the PH strap (as illustrated in FIGS. 2, 5-8).

### Use of this Invention

All needs to happen is a baby who needs the pacifier to be rested, calm down and stop crying. Get the PH strap and a pacifier, run the pacifier handle through the hole/slot of the PH strap.

Now place the pacifier in baby's mouth and grab the ends of the strap, go around the lower part of the back-of-baby's head (or the upper part of the backside of the neck) and attach the two Velcro ends to each other. Make certain that the strap is not very tight to bother or place tension on the baby's face and back of neck. It is suppose to huddle for the baby to move his/her jaw and cheeks, but nut too loose for the pacifier to be pushed out. The users must use his/her adult “best-Judgment” before attaching the two ends of this PH strap. The 2-3″ of Velcro sewed to each end is a mean for the user to adjust the strap so that it would be a perfect fit without being tight.

Other Benefits of these Pacifier Holder (PH, PHB, PHC, PHD) and the Attachment:

Unlike the inventor's experiences observed at the professional care provider location where the babies are “supposed to” be cared for “safely” (the day care centers), and as described above and below in the Background, the inventions of the Pacifier Holder (PH, PHB, PHC, PHD) and the attachments have many advantages.

0001. NO discomfort and deformity: PH, PHB, PHC are NOT attached to any part of the baby's body (tissues). The loop around the ear (the old design) is not only discomforting to an adult (let alone a baby), but also deforms the ears of a child at the stage when the baby is just starting to form properly and any mishaps will create an issue, problem, illness and/or deformation. In PHD, the ear-loop is made of soft thin fabric; hence, there will not be any discomfort or it may be minimal.

0002. NO risk of short or long term (future harm). The experiences observed at the professional care provider centers (day cares), in which they covered babies' faces with blanket or other hefty items, creates several risks. These actions can cause heat exhaustion, suffocation, cause paranoia for the baby or many other disadvantages (that may show later in the baby's life), since these items shift as the baby moves his/her head repeatedly, unconscionably and inconsistently. They would heat up the baby's face beyond a normal natural temperature, as God intended, making the baby sick or cause many other issues.

0003. Unit weight: This unit will remain in place over the baby's face around the mouth only and the attachment will become part of the baby's closed eyes. Neither of the units are heavy or bulky, yet attached and move exactly the same amount as the baby moves his/her face; therefore, they remain in their designated place as intended. This weights less 2 oz (depending on weight of fabric). However, this weight is distributed all around the baby's face and neck; therefore there is no weight impact.

0004. No pressure, stress or weight on baby: Being just a layer of fabric with a very little weight without a frame, makes it comforting to know—it won't put any pressure, heaviness or stress on a baby's face or eyes. Again unlike the U.S. Pat. No. 4,969,894 A invention.

0005. Productivity: This also allows the care provider (Parents) feel comfortable that he/she does not have to run to the baby's aid as frequently (each time that the baby cries for a pacifier), therefore, increases the Late provider's productivity in whatever other task being performed.

0006. Safety: (a) The rushing of care provider may cause the provider to create a dangerous condition (while rushing to baby's aid). (b) it is a safe way to reduce a baby from being stressed or cry frantically because of the missing element (pacifier).

0007. Market Cost: The material used in this invention would not cost more than a dollar and with its labor and marketing, one should be easily and cheaply produce and reproduce this design and provide it to the mass market at a minimal cast.

ALSO the Eye-Cover:

0008. As described above, this eye-cover is very easy to use, it is attached and detached by using the velcro or sticky connectors of the PH strap and this unit.

It acts as a blindfold over baby's eyes (without having the tension of pressure, to help baby relax and sleep quickly. This invention is attached to the invention of PH, PHB, PHC, PHD through two pieces of stick tapes or Velcro. The eye piece can be folded back (to fall over the chin and act as a cleaning tissue) or removed/detached from the face completely, when it is not needed.

0001. The present invention relates to the concept of having a pacifier holder strap (PH) or harness, in particular a stretchable retainer to very gently and yieldably hold a pacifier gently in a baby/infant mouth.

0002. The pacifier holder strap (PH) a.k.a stretchable retainer (PH) of claim 1, consists of a thin, flexible, stretchable, soft strip of fabric (varieties of designs and shapes) with a preferated hole in its center to hold a pacifier in the baby's mouth as long as needed.

0003. The stretchable retainer (PH) of claim 2, can be easily be removed by the care giver when not needed or needs to be replaced.

0004. In the stretchable retainer (PH) of claim 3, if the baby gets tired of sucking on the pacifier, he/she can simply and easily push the pacifier out of his/her mouth using tong or lips—while the PH strap is still retained around the baby's face (below his/her bottom lip). In this instant, the pacifier will stay out of baby's mouth, but immediately close to his lips for retraction. Then, if the baby desires to have it back, he/she can simply move his lips and play with the pacifier and have it have in his/her mouth OR easily push the pacifier into the mouth with his/her hands. This entire was repeatedly observed as the inventor was testing the invention with four (4) separate babies (to include two of his own).

0005. The stretchable retainer (PH) of claim 4, can be designed in different styles as illustrated by PH, PHB, PHC, PHD in the Specification/Detailed Description of the Invention section.

0006. Either method and design of the stretchable retainer (PH) of claim 5, (as described Specification section) will have the same Statement of Claim which consists of:
a. The stretchable retainer (PH) of claim 5, is designed to give comfort, peace and rest to both the baby using the unit and the parents/care-giver of the baby without any of them getting stressed;
b. The stretchable retainer (PH) of claim 5, is designed to reduce baby's refluxes and increasing baby's digestive activity,
c. The stretchable retainer (PH) of claim 5, is designed to Helps baby itching and scratching gum issues during the early stage of teething;
d. The stretchable retainer (PH) of claim 5, is designed to assist baby's continuous gum development even at the time the baby is sleep;
e. The stretchable retainer (PH) of claim 5, is designed to give parents of new baby an extended rest so that they do not have to run back and forth to place pacifier in a crying baby's mouth;
e. The stretchable retainer (PH) of claim 5, is designed to protect a baby from inserting other objects that may also be unsanitary into his/her mouth;
f. The stretchable retainer (PH) of claim 5, is designed to consider that, if a pacifier falls out of baby's mouth, it is kept close to his/her mouth and would not fall on ground/floor, where it would make the child be exposed to an unsanitary pacifier unless the pacifier is cleaned before it is put back into baby's mouth again (delaying time to stop the pacifier desiring and crying baby's stress);
g. The stretchable retainer (PH) of claim 5, has advantages, in which One major Advantage is that the pacifier retainer/strap is made of materials that is so flexible and stretchable that if desired or necessary, the pacifier could be removed by the infant/baby without anyone's assistance and would allow the baby to easily breath, cry, smile or even make noise without gagging;
h. The stretchable retainer (PH) of claim 5, is designed to fit any size pacifier
i. The stretchable retainer (PH) of claim 5, is designed to be very light weight, portable on baby's face anywhere, and
j. The stretchable retainer (PH) of claim 5, is designed to be adjustable to fit any size baby.
